(19) United States
(12) Patent Application Publication
(10) Pub. No.: US 2005/0176067 A1
(43) Pub. Date: Aug. 11, 2005

(54) Biosensor for determining an allergen with operating procedures

(76) Inventors: Maximilian Fleischer, Hoehenkirchen (DE); Corinna Haindl, Muenchen (DE); Hans Meixner, Haar (DE); Elfriede Simon, Muenchen (DE)

Correspondence Address: HARNESS, DICKEY & PIERCE, P.L.C., P.O.BOX 8910, RESTON, VA, 20195, US

(21) Appl. No.: 11/049,953

(22) Filed: Feb. 4, 2005

(30) Foreign Application Priority Data
Feb. 5, 2004 (DE) 10 2004 005 711.7

(51) Int. Cl. G01N 33/53; G01N 33/537; G01N 33/543; C12M 1/34

(52) U.S. Cl. 435/7.1; 435/287.2

(57) ABSTRACT

A biosensor is for detecting an allergen-specific immunoglobulin E (IgE) via antigen/antibody coupling. The biosensor includes a silicon substrate and at least one interdigital electrode pair structure applied to the silicon substrate with a gap between the electrode pairs of a maximum of 1.0 μm. A counter electrode is further applied to the silicon substrate. The biosensor also includes a reference electrode. Additionally, coatings are included on the biosensor. A first coat is made from protein coating at least the interdigital electrode structure; a selective second coat is made from protein applied over the first coat, the second coat containing a selected captor antibody; and a third coat is applied over the first coat, which contains the allergen which can couple to the captor antibody. A sensor signal can be readout at the interdigital electrode structure, if an allergen-specific immunoglobulin E (IgE) from a sample of a human blood serum in contact with a biosensor couples to the allergen present on the sensor surface. Further, an enzymatic release of a redox reactive molecule takes place on the sensor surface via an enzyme-marked detection antibody similarly coupled to the allergen-specific immumoglobulin E (IgE).

The present application hereby claims priority under 35 U.S.C. §119 on German patent application number DE 10 2004 005 711.7 filed Feb. 5, 2004, the entire contents of which are hereby incorporated herein by reference.

### BACKGROUND OF THE INVENTION

The number of allergic diseases has drastically risen over the last few years as a result of an increasing environmental impact. Thus, the demand for methods to detect this immunological dysfunction has also increased.

With the onset of an allergy, the reaction of the body's own immune system results in symptoms such as erythema, stinging eyes, swelling of the skin and/or mucosa and breathlessness right through to asthma attacks. In addition to the outwardly visible allergic reaction, considerable changes in the immune system of the body of an allergy sufferer occur. Contact with an allergen results in special types of antibodies such as immunoglobulin E (IgE) being found in the blood in higher concentrations, which are not present in healthy people without allergies.

On the basis of the specifity, in other words by way of the reaction and/or binding behaviour, the antibodies can be identified. As such, the allergen generates the formation of the IgE, each IgE only binding itself to the allergen which was responsible for its formation.

There are two different approaches used in detecting the allergen:

1. Examination of the Body's Own Outwardly Visible Reaction:

In order to diagnose an allergy in a patient, an ‘in vivo’ test is generally carried out, i.e. a test directly on the patient, the so-called ‘prick test’, whereby the patient is brought into contact with various allergy inducing agents and an allergic reaction based on the subsequent immune reaction is identified, in other words the reaction of the skin to the allergen. Various diluted allergens are injected under the skin surface (arm or back) at marked points. Finally the skin reaction of the test person is monitored over several days in respect of redness or the formation of welts and the extent of the allergic reaction is evaluated.

This method is painful for the patient and can therefore not be repeated often. Furthermore, the number of allergens which can be tested is restricted.

2. Examination of the Immune System—Immunological Tests:

The market leader in the current ‘in vitro’ allergy test, in other words the diagnostics in vitro with a serum sample from the patient, is the CAP test from the Pharmacia company. This relates to an optical readout principle. A carrier polymer, such as a CNBr-activated cellulose derivative for example allows various allergens to be adsorptively immobilised. Specific IgE present in patient serum binds itself to the position with the corresponding allergen and is detected by means of anti-human IgE. Anti-human IgE is bound to β-galactosidase or ¹²⁵I. The bound IgE is detected photometrically in the case of the enzyme immunoassays, or correspondingly on the basis of the radioactive isotope decay. The disadvantage of this test is the large quantity of serum required, amounting to more than 10 ml and the high outlay in apparatus and time of more than 30 minutes.

Further immunological tests available on the market are based on the detection of a colour response to a simple cellulose test strip, such as allergoscreen from Ganzimmun or allergodip from Allergopharma. This detection method is not overly sensitive and provides a first qualitative statement about the existence of allergic diseases, but is however not suitable for clinical diagnostics. [IV].

### SUMMARY OF THE INVENTION

An object of an embodiment of the invention is to provide a biosensor for detecting an allergy in a person and/or an antibody in his/her blood, by way of a biosensor and an operating procedure. As such, a plurality of allergies can preferably be detected and differentiated with a high level of sensitivity.

An object may be based on the knowledge that,
antigen/antibody reactions can be marked using a detection antibody coupled to an enzyme,
specific proteins such as protein A, G, G′ or L for example cause an intentional binding to the antibody molecule,
a redox recycling can be induced by enzymatic separation of pAP (para aminphenol) for example, to IDS, and/or
a counter electrode and a reference electrode are advantageous for the electrochemical readout of the redox cycling.

The technology of the biochip base may be known from: [I], [II], [III].

### BRIEF DESCRIPTION OF THE DRAWINGS

Exemplary embodiments are described below with reference to exemplary schematic drawings not restricted to the invention, in which;

FIG. 1 shows a schematic structure of the biological coating on a gold electrode and a diagram of the detection function;

FIG. 2 shows electrode processes on the interdigital electrodes with the amperometric readout reaction using p-aminophenol;

FIG. 3 shows a schematic structure of a measuring station in for reading out the sensor chip signal;

FIG. 4 shows the schematic representation of the sensor chip used, with reference electrodes, a plug contact for micro poteniostat and enlargement of the silicon chip using interdigital structures and counter electrodes;

FIG. 5 shows a schematic structure of an interdigital structure; and

FIG. 6 shows a typical sensor signal during the measurement of a positive and a negative sample considering as example the household dust mite allergy.

### DETAILED DESCRIPTION OF THE EXEMPLARY EMBODIMENTS

Structure of a biosensor which can be read-out electronically to detect the specific immunoglobulin E (IgE), which appears as an antibody in the serum of the person with an allergy.

The biochip and/or the biochip array are characterized by the following characteristics:

The silicon chips of approximately 4.5 mm*6 mm in size which are contacted with the readout device by way of a printed circuit board each carry a plurality, particularly between 12 and 26, of circular areas with interdigital structures of a diameter of 200 μm to 400 μm, depending on their layout. The comb-like electrode finders 13 of the interdigital structures have a width of 1 μm and a gap of a maximum of 1.0 μm. In addition, a counter electrode 11 made from gold is arranged on the chip. In this case, the Ag/AgCl reference electrode 9, against which the electrode potential is set, is not found on the chip, but is integrated in the flow system.

The allergen 6 is immobilized on the gold electrode 3 of the sensor chip 10. This is done with the aid of a first coat made from protein, comprising protein A, G G′ or L, in combination with subsequent coat with an immobilised so-called captor antibody 5, to which the allergen 6 found in the third coat is bound.

In the case of an allergic disease, the allergen-specific IgE present in the serum of an allergic patient will bind itself to the allergen 6 immobilised at the surface on the biochip 10 by means of antigen/antibody coupling. This binding is detected by way of an enzyme-marked second antibody, the detection antibody 7.

The sensor signal is read out electrically at the sensor surface 1, using a multichannel potentiostat after an enzymatic release of a redox reactive molecule, p-Aminophenol for example.

The type of allergy, i.e. against which allergen the patient shows an allergic reaction can be extremely quickly, efficiently and cost-effectively determined by detecting the IgE developing in the patient serum, with the aid of an electrical biochip.

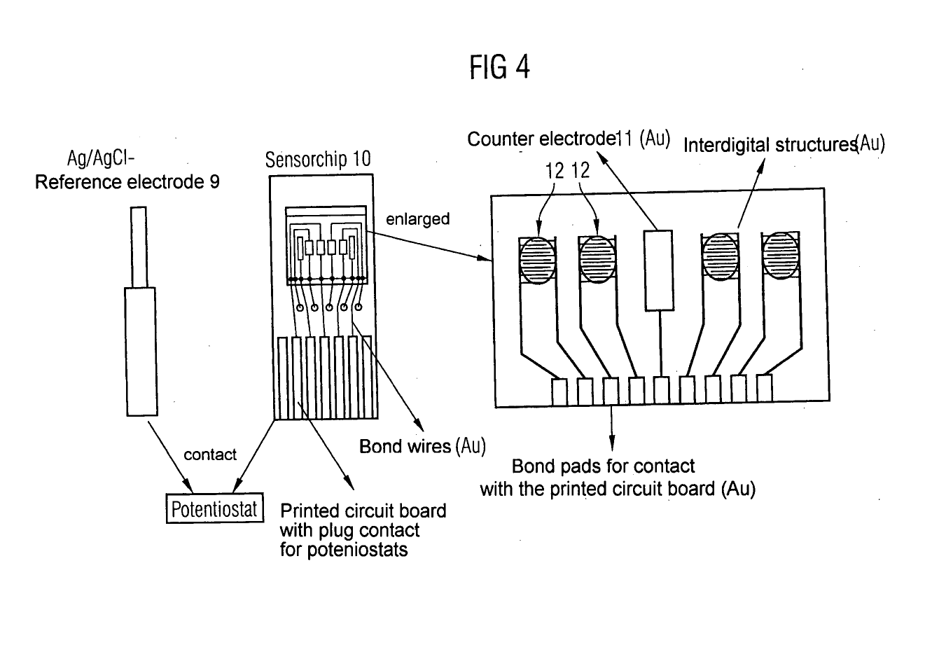

The biosensor used is silicon-based with gold interdigital structures 12 [I, II, III]. FIGS. 4 and 5 schematically show the interdigital electrode pair structures featuring gaps in the micrometer range. The electrode pair gap must be less than 1 μm.

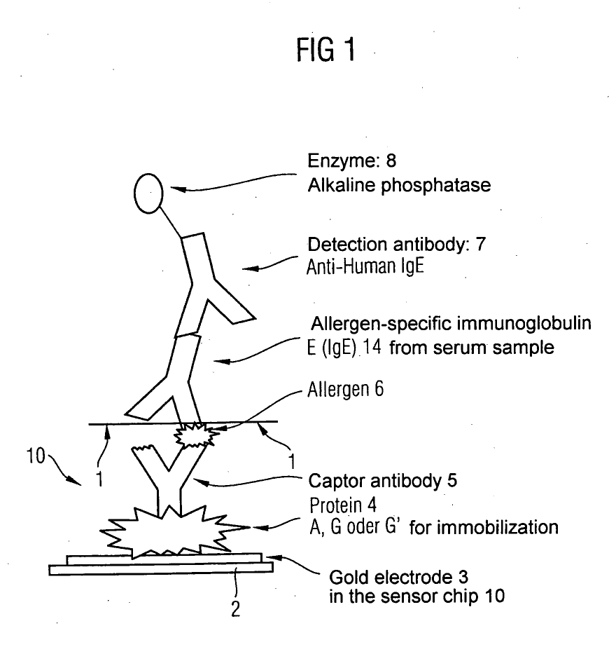

The allergen is immobilised on the gold interdigital structures of the biochip, by way of the following coat structure, see FIG. 1.

The first step is to produce a protein base coating, which takes place by way of unspecific adsorption of protein A, G, G′ or L for example, directly onto the gold surface of the interdigital structure. The choice of protein depends on the captor antibody 5.

The second step is the explicit binding of the captor antibody 5 to the first protein base coating.

In the third step, the corresponding allergen 6 is selectively bound to the captor antibody 5, thus providing a defined and highly selective chip surface.

FIG. 1 shows a schematic structure of the biological coat on the gold electrode 3.

By immobilising different allergens at different positions on a sensor array, whereby several hundred positions are possible, the sensor surface becomes a analysis landscape which can be used with a plurality of different allergens, by way of the above described coating of the biochip (protein coating/captor antibody/allergen).

The coated biosensor chip is incubated with patient serum in order to implement the ‘in vitro’ allergy test. With a positive reaction, the allergen-specific immunoglobulin E 14 present in the serum binds itself to the corresponding allergen 6 immobilised on the chip, by way of an antigen/antibody reaction.

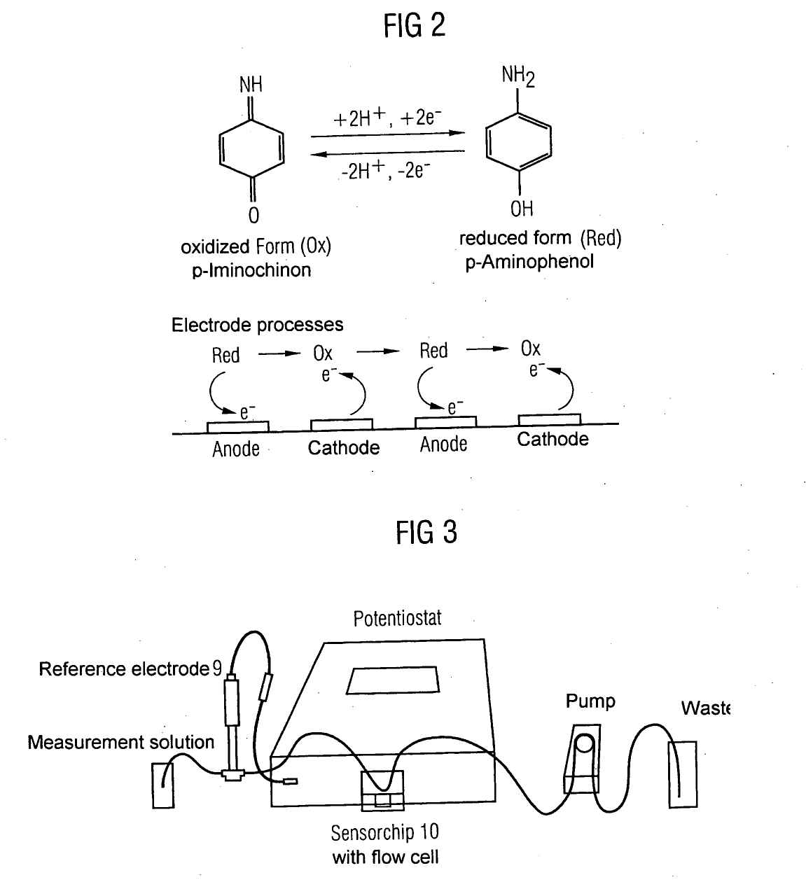

The biochip is bonded with a multichannel potentiostat for an electrical (amperometric) readout, FIG. 2, and correspondingly directed to the substrate matching the enzyme 8 used, for example p-aminophenylphosphate, by way of a fluid system in accordance with FIG. 2. The substrate is converted into redox active p-aminophenol by way of the enzyme alkaline phosphatase coupled to the detection antibody 7. A specific current, for example 350 mV, applied between the electrode of the biochip subjects the released p-aminophenol to a reduction and subsequent oxidisation at the cathode and anode of the interdigital electrodes corresponding to FIG. 2, whereby a change in the sensor signal is produced as a measurable voltage in the nA range.

FIG. 2 shows the electrode processes on the interdigital electrode pair structure with the amperometric readout reaction using p-aminophenol.

Another enzyme-linked detection antibody 7 can be used to detect the specific immunoglobulin IgE, whereby instead of p-aminophenylphosphate another ‘suitable’ substrate must develop to react with the used enzyme 8,. Further suitable enzyme-bound antibodies and/or their substrates are: B galactosidase, coupled to the detection antibody and p-aminophenyl-β-D-galactopyranoside as a substrate.

A total of three antigens/antibody couplings are used in this detection. The first is arranged between the captor antibody 5 and the allergen 6 immobilised on the biosensor, the second arranged between allergen 6 and the allergen-specific immunoglobulin E 14 and the third arranged between the allergen-specific immunoglobulin E 14 and the detection antibody.

Major benefits of embodiments of the invention include the fact that disadvantages prevailing with the conventional allergy test can be reduced or even minimized with the aid of this amperometric allergy biochip.

The outstanding features of the test principle presented are that by using correspondingly suitable sensor chips and potentiostats, a large number of allergens, for instance more than 100 can be read out in a very short space of time (10 minutes), and with a very low quantity of serum (˜1μ1).

The patient is disturbed only once in order to take one drop of blood, this being possible without an injection and thus being considerably more pleasant for the test subject, in comparison with current ‘in vitro’ allergy tests which require a blood sample of several millilitres.

The test can be repeated several times over a short period of time.

Sample preparation such as cell disruption, cleaning of the serum for instance is not necessary.

The use of an electrical biochip allows the disadvantages of optical methods to be reduced, said methods above all involving a major outlay in equipment.

An extremely cost-effective and simple allergy test can be produced using this biochip.

The multi structure of the biochip also shows no matrix effects which would otherwise lead to a more complex sample preparation thereby considerably increasing the test duration.

Relevant test setups for the invention are as follows:

a) Structure of the Measuring Station

FIG. 3 shows the schematic structure of the measuring station for reading out the sensor chip.

b) Structure of the Sensor Chip System

FIG. 4 shows a schematic representation of the sensor chip used, with reference electrodes, plug contact for micro potentionstats and enlargement of the silicon chip 10 with an interdigital structure 12 and counter electrodes 11.

c) Interdigital Structures

FIG. 5 shows a schematic structure of an interdigital electrode pair structure.

Considering as an example the household dust mite allergens, ‘Der p2’, the operability of the measurement principle introduced was tested in a practical application as an in vitro allergy test. In this test, only two positions (2×2 interdigital structures) of a chip were firstly coated in parallel and read out. The allergen was bound to the electrode surface by means of protein G′ and the monoclonal antibody 1D8 (Anti-Der p2). Finally position 1 was incubated with a blood sample which was already positively tested in another method and position 2 with a negative.

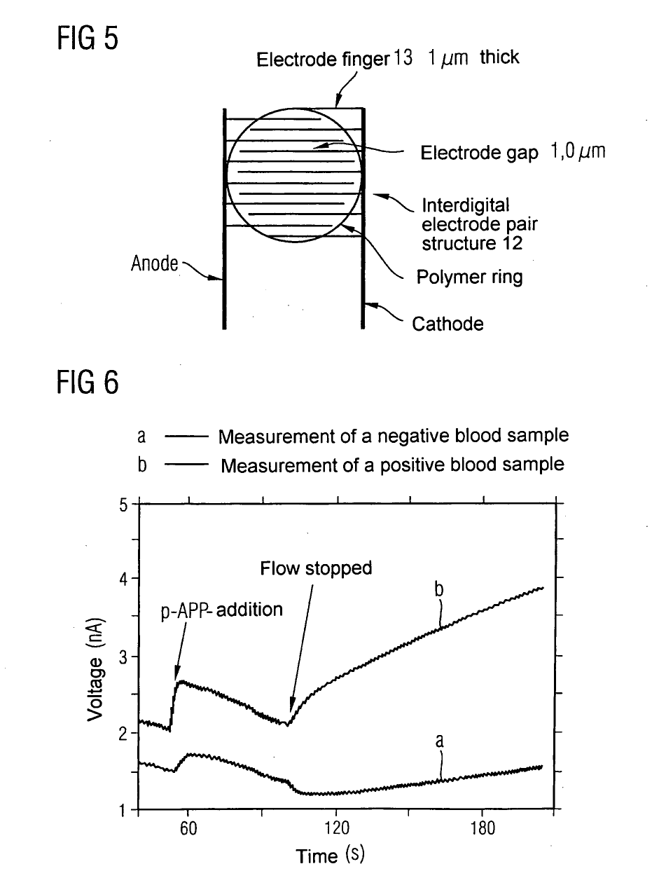

By adding the detection antibody 7 (Anti-Human IgE), in the case of the positive sample, Der p2-specific IgE 14 is marked with enzyme 8 and the chip 10 is directly read out. For this the sensor is built into the flow cell, bonded with the multichannel potentiostat and initially rinsed with buffers. After the addition of the p-aminophenylphosphate, there is a wait of around 30 seconds and then the flow is stopped, see FIG. 6.

FIG. 6 shows a typical sensor signal during the measurement of a positive and a negative sample, using the household dust mite allergy as an example. FIG. 6 shows that the sensor signal of the positive sample lies above the signal of the negative sample during the entire measurement process. Experience has shown that material-specific deviations can arise between the individual positions. Thus, both signals do not lie at 0 nA as expected, even before the substrate is added.

Since the p-aminophenol generated at the positive position is also subject to an active transportation within the cell when the flow is switched on, the behaviour of the respective positions on flow stop is taken into account in order to evaluate the sensor signal. The signal increases at the flow stop in the case of a positive signal response. The reason for this lies in the increasing concentration of p-aminophenol at the position occupied by the enzyme, which is not removed through the flow. At the same time, the developing p-aminophenol is not transported to the adjacent negative position, thereby resulting in a disruption to the concentration and also to the measurable voltage signal when the flow is stopped.

This method has proven to be reliable in measuring blood samples, since no incorrect-positive measurement results were achieved, in other words, a positive blood sample was always present in the event of a positive sensor signal on flow stop.

Exemplary embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

### LITERATURE

[I] Hintsche, R. and M. Paeschke (2000). Detektion von Molekülen und Molekülkomplexen. Patentschrift DE 19610115C2. Deutschland. (Detection of molecules and molecule complexes. Patent application DE 19610115 C2. Germany).
[II] Hintsche, R., M. Paeschke, et al. (1997). Microbiosensors using electrodes made in Si-technology. Frontiers in Biosensorics 1—Fundamental Aspects: 267-283.
[III] Paeschke, M., F. Dietrich, et al. (1996). “Voltammetric Multichannel Measurements Using Silicon Fabricated Microelectrode Arrays.” Electroanalysis 8 (10): 891-898.
[IV] Schlenvoigt, G et al. (1997) Allergodip—ein neuer Streifentest zum Nachweis spezifischer IgE Antikörper in Seren von Allergikern im Vergleich mit CAP, Hautpricktest und der Klinik Allergologie, Jahrgang 20, Nr. 10, 512-518. (Allergodip—a new strip test for detecting specific IgE antibodies in the serum of allergy sufferers in comparison with CAP, skin prick test and the allergology clinic, age-group 20, No. 10, 512-518).

1. Biosensor for determining an allergen-specific immunoglobulin E (IgE) using antigen/antibody coupling, comprising:
a silicon substrate;
at least one interdigital electrode pair structure applied to the silicon substrate, wherein a gap between the electrode pair is a maximum of 1.0 μm;
a counter electrode applied to a silicon substrate;
a reference electrode;
a first coat made from protein covering at least the interdigital electrode structure;
a selective second coat made from protein applied over the first coat, containing a selected captor antibody;
a third coat applied over the second coat, containing the allergen which coupleable to the captor antibody, whereby a sensor signal is readable at the interdigital electrode pair structure if one of the samples of human blood serum in contact with the biosensor couples an allergen-specific immunoglobulin E (IgE) to the allergen is present on the sensor surface, and wherein an enzymatic release of a redox reactive molecule takes place at the sensor surface via an enzyme-marked detection antibody coupled similarly with the allergen-specific immunoglobunlin E (IgE).

2. Biosensor according to claim 1, wherein the first protein coat is made from at least one the proteins A, G, G′ and L.

3. Biosensor according to claim 1, wherein the captor antibodies exhibit a directed binding to the protein of the first coat in order to increase the selectivity of the second coat.

4. Biosensor according to claim 1, wherein a signal is detected via at least one of an alternating current and a cyclical voltammetry, instead of the amperometric readout using redox recycling.

5. Biosensor according to according to claim 1, wherein the biosensor is coupled to a potentiostat in order to read out the sensor signal.

6. Biosensor according to according to claim 1, wherein the serum sample to be analyzed is provided as fluid on the surface of the biosensor, via a flow system.

7. Biosensor according to according to claim 1, wherein interdigital electrode structures and counter electrodes are made from gold.

8. Biosensor according to according to claim 1, wherein the reference electrode represents an Ag/AgCl reference.

9. Biosensor according to according to claim 1, wherein a reference electrode is integrated onto a biosensor.

10. Method for operating a biosensor for detecting allergen-specific immunoglobulin E (IgE) via antigen/antibody coupling, comprising:
manufacturing a first coat on a silicon substrate, made from a protein A, G, G′ or L, at the same time as coating interdigital electrode pair structures, found directly on the surface of the silicon substrate;
manufacturing a second coat on a protein based coating containing a captor antibody attuned to the protein of the first coat, selected such that this couples with the sought-after allergen;
manufacturing a third coat which contains the allergen;
bonding the sensor surface using a blood serum to be analyzed, whereby an allergen-specific immunoglobulin E (IgE) contained in a blood serum is selectively bindable to the allergen of the upper coating;
marking the allergen-specific immunoglobulin E (IgE) by use of a detection antibody coupled to an enzyme and simultaneously coupled to the allergen-specific immunoglobulin E (IgE); and
reading out a sensor signal at the interdigital electrode pair structures via redox recycling, wherein the enzyme-bound detection antibody activates an enzymatic release of a redox reactive molecule on the sensor surface.

11. Biosensor according to claim 2, wherein the captor antibodies exhibit a directed binding to the protein of the first coat in order to increase the selectivity of the second coat.

12. Biosensor according to claim 2, wherein a signal is detected via at least one of an alternating current and a cyclical voltammetry, instead of the amperometric readout using redox recycling.

13. Biosensor according to according to claim 2, wherein the biosensor is coupled to a potentiostat in order to read out the sensor signal.

14. Biosensor according to according to claim 2, wherein the serum sample to be analyzed is provided as fluid on the surface of the biosensor, via a flow system.

15. Biosensor according to claim 3, wherein a signal is detected via at least one of an alternating current and a cyclical voltammetry, instead of the amperometric readout using redox recycling.

16. Biosensor according to according to claim 3, wherein the biosensor is coupled to a potentiostat in order to read out the sensor signal.

17. Biosensor according to according to claim 3, wherein the serum sample to be analyzed is provided as fluid on the surface of the biosensor, via a flow system.

18. Biosensor according to according to claim 4, wherein the biosensor is coupled to a potentiostat in order to read out the sensor signal.

19. Biosensor according to according to claim 4, wherein the serum sample to be analyzed is provided as fluid on the surface of the biosensor, via a flow system.

20. Biosensor according to according to claim 5, wherein the serum sample to be analyzed is provided as fluid on the surface of the biosensor, via a flow system.
